(19) United States
(12) Patent Application Publication
(10) Pub. No.: US 2012/0277695 A1
(43) Pub. Date: Nov. 1, 2012

(54) TRANSDERMAL DELIVERY PATCH

(76) Inventors: Jeremy Cottrell, Caulfield South (AU); Giacinto Gaetano, South Melbourne (AU); Mahmoud El-Tamimy, Meadow Heights (AU); Nicholas Kennedy, Boronia (AU); Paul David Gavin, Chadstone (AU)

(21) Appl. No.: 13/501,499

(22) PCT Filed: May 18, 2010
(86) PCT No.: PCT/AU10/00580

§ 371 Date: Apr. 12, 2012

(30) Foreign Application Priority Data
Mar. 30, 2010 (US) 60319002

(51) Int. Cl. A61M 35/00; B29C 39/38; A61K 47/32

(52) U.S. Cl. 604/304; 514/772.4; 264/330

(57) ABSTRACT

A composition suitable for use in a transdermal delivery patch for administration of an opioid, the composition comprising a phosphate compound of tocopherol and a polymer carrier.

### TECHNICAL FIELD

The present invention relates to a transdermal delivery patch for administration of therapeutic compounds. More specifically, the present invention relates to a transdermal delivery patch for administration of opioids.

### BACKGROUND

In this specification where a document, act or item of knowledge is referred to or discussed, this reference or discussion is not an admission that the document, act or item of knowledge or any combination thereof was at the priority date, publicly available, known to the public, part of common general knowledge; or known to be relevant to an attempt to solve any problem with which this specification is concerned.

Drug delivery is the method or process of administering a pharmaceutical compound to achieve a therapeutic effect in humans and animals.

Drug delivery technologies have been developed to improve bioavailability, safety, duration, onset or release, of the pharmaceutical compound.

When developing drug delivery technologies, problems likely: to be encountered include compatibility of the drug delivery system and the pharmaceutical compound, maintaining an adequate and effective duration, potential for side effects, and meeting patient convenience and compliance. As a consequence, many drug delivery technologies fall short of desired improvements and requirements.

Accordingly, there is still a need for alternate drug delivery systems that effectively deliver drugs.

### SUMMARY

It has surprisingly been found that opioids can be effectively administered using a transdermal delivery patch.

According to a first aspect, there is provided a composition suitable for use in a transdermal delivery patch for administration of an opioid, the composition comprising a phosphate compound of tocopherol and a polymer carrier.

In one embodiment, the transdermal delivery patch is a matrix patch.

A second aspect provides use of a phosphate compound of tocopherol and a polymer carrier as a matrix layer in a transdermal delivery patch for administration of an opioid.

The phosphate compound of tocopherol may be selected from the group consisting of mono-(tocopheryl)phosphate, mono-(tocopheryl)phosphate monosodium salt, mono-(tocopheryl)phosphate disodium salt, mono-(tocopheryl)phosphate monopotassium salt, mono-(tocopheryl)phosphate dipotassium salt, di-(tocopheryl)phosphate, di-(tocopheryl)phosphate monosodium salt, di-(tocopheryl)phosphate monopotassium salt, or a mixture thereof. These phosphate compounds may be derived from the alpha, beta, gamma or delta form of tocopherol, or a combination thereof.

The composition, or matrix layer, may comprise a phosphate compound of tocopherol in an amount within the range of about 0.01% w/w to about 10% w/w, about 0.1% w/w to about 5% w/w, about 0.5% w/w to about 2% w/w or to about 3% w/w, of the total concentration of the matrix layer. In one embodiment, the phosphate compound of tocopherol is present in an amount of about 1% w/w to about 1.5% w/w of the total concentration of the matrix layer.

The polymer carrier may comprise natural and synthetic polymers, co-polymers, or terpolymers. Preferred polymer carriers that are suitable for use in the composition, or matrix layer, include polyvinyl pyrrolidone (e.g. PVP K90, MW 360,000 Da), polysiloxanes and polymethyl methacrylate (e.g. Eudragit E100). The composition, or matrix layer, may comprise a polymer carrier in an amount of from about 20% w/w up to about 90% w/w, from about 30% w/w up to about 80% w/w, from about 55% w/w up to about 65% w/w, of the total weight of the composition, or matrix layer.

The polymer carrier may also comprise inert carrier components selected from the group consisting of anti-tacking agents, tackifiers, and plasticizers.

Inert carrier components may be present in the composition, or matrix layer, in an amount of from 0.001% w/w up to about 50% w/w, up to about 40% w/w, from up to about 30% w/w, of the total weight of the composition, or matrix layer.

A third aspect provides a transdermal delivery patch for administration of an opioid comprising (i) a backing layer, and (ii) a matrix layer, which comprises a phosphate compound of tocopherol and a polymer carrier (as defined above), and an opioid.

A fourth aspect provides use of a matrix patch for transdermal delivery of an opioid, the matrix patch comprising (i) a backing layer and (ii) a matrix layer, which comprises a phosphate compound of tocopherol and a polymer carrier (as defined above), and an opioid.

The opioid may be selected from the group consisting of morphine, codeine or thebaine; hydromorphone, hydrocodone, oxycodone, oxymorphone, desomorphine, diacetylmorphine (heroin), nicomorphine, dipropanoylmorphine, benzylmorphine or ethylmorphine; fentanyl, pethidine, methadone, tramadol or dextropropoxyphene; endorphins, enkephalins, dynorphins, or endomorphins.

The opioid may also be selected from the group consisting of opioid receptor agonists including morphine, depomorphine, etorphine, heroin, hydromorphone, oxymorphone, levorphanol, methadone, levomethadyl, meperidine, fentanyl, sufentanyl, alfentanil, codeine, hydrocodone, oxycodone, and mixtures thereof; opioid receptor antagonists including naloxone and naltrexone; opioid receptor mixed agonist-antagonists including buprenorphine, nalbuphine, butorphanol, pentazocine, and mixtures thereof; and, ethylketocyclazocine.

The opioid may also be selected from the group consisting of codeine, morphine, thebaine and oripavine; diacetylmorphine (heroin), dihydrocodeine, hydrocodone, hydromorphone, nicomorphine, desmorphine, ethylmorphine, dipropanoylmorphine, oxycodone and oxymorphone; fentanyl, alphamethylfentanyl, alfentanil, sufentanil, remifentanil, carfentanyl and ohmefentanyl; pethidine (meperidine), ketobemidone, MPPP, allylprodine, prodine and PEPAP; propoxyphene, dextropropoxyphene, dextromoramide, bezitramide, piritramide, methadone, dipipanone, levomethadyl acetate (LAAM), difenoxin, diphenoxylate and loperamide; dezocine, pentazocine and phenazocine; buprenorphine, dihydroetorphine and etorphine; butorphanol, nalbuphine, levorphanol and levomethorphan; lefetamine, meptazinol, tilidine, tramadol and tapentadol; nalmefene, naloxone and naltrexone; and pharmaceutically-acceptable salts, prodrugs, or derivatised compounds thereof.

In a preferred embodiment, the opioid is oxycodone or dihydrohydroxycodeinone (oxycodone base).

The opioid may be present in an amount of from about 0.1% w/w up to about 30% w/w, up to about 20% w/w, up to about 10% w/w, of the total concentration of the composition, or matrix layer. In one embodiment, the composition, or matrix layer, will have an opioid concentration of about 4.5% w/w to about 5.5% w/w of the total concentration of the composition, or matrix layer.

Preferably the backing layer is occlusive.

A fifth aspect provides a method for preparing a transdermal delivery patch for administration of an opioid comprising the steps of:
(i) combining a polymer carrier and optional inert carrier components with a suitable solvent;
(ii) combining (i) with a dispersion comprising a phosphate compound of tocopherol and an opioid;
(iii) stirring (ii) until complete homogenisation is achieved;
(iv) placing (iii) in a mould comprising a suitable backing layer; and
(v.) drying the compositions in the mould by heating them up to about 90° C. for about 0.5 to about 24 hours. Preferably, the drying is conducted at a temperature of 75° C.

### DETAILED DESCRIPTION

The present invention relates to a composition suitable for use in a transdermal delivery patch for administration of an opioid, the composition comprising a phosphate compound of tocopherol and a polymer carrier. The composition, or matrix layer, may form part of a transdermal delivery patch. It has been surprisingly found that a transdermal delivery patch comprising this matrix layer can effectively administer opioids.

### Phosphate Compound of Tocopherol

The composition, or matrix layer, comprises a phosphate compound of tocopherol.

Vitamin E exists in eight different forms, namely four tocopherols and four tocotrienols. All feature a chroman ring, with a hydroxyl group that can donate a hydrogen atom to reduce free radicals and a hydrophobic side chain which allows for penetration into biological membranes. Such derivatives of vitamin E may be classified as “hydroxy chromans”. Both tocopherols and tocotrienols occur in alpha, beta, gamma and delta forms, determined by the number and location of methyl groups on the chroman ring. The tocotrienols differ from the analogous tocopherols by the presence of three double bonds in the hydrophobic side chain. The various forms of vitamin E are shown by Formula (I):

| | |
|---|---|
| | (I) |
| 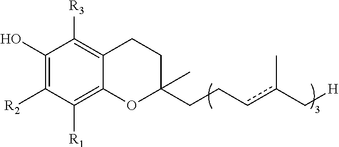 | |

| | R₁ | R₂ | R₃ |
|---|---|---|---|
| α-tocopherol | CH₃ | CH₃ | CH₃ |
| α-tocotrienol | | | |
| β-tocopherol | CH₃ | H | CH₃ |
| β-tocotrienol | | | |
| γ-tocopherol | H | CH₃ | CH₃ |
| γ-tocotrienol | | | |
| δ-tocopherol | H | H | CH₃ |
| δ-tocotrienol | | | |

In the present invention, tocopherol in any of the four forms may be used. The alpha form of tocopherol is preferred.

The term “phosphate compound” refers to phosphorylated tocopherol, where a covalent bond is formed between an oxygen atom (typically originating from a hydroxyl group) of the tocopherol compound and the phosphorous atom of a phosphate group (PO₄).

The phosphate compound may be a phosphate mono-ester, phosphate di-ester, phosphate tri-ester, pyrophosphate mono-ester, pyrophosphate di-ester, or a salt or derivative thereof, or a mixture thereof. The di- and tri-esters may comprise the same tocopherol form or different tocopherol forms.

The “salts” include metal salts such as alkali or alkaline earth metal salts, for example sodium, magnesium, potassium and calcium salts. Sodium and potassium salts are preferred.

The “derivatives” include phosphate compounds where one or more phosphate protons are replaced by a substituent. Some non-limiting examples of derivatives include phosphatidyl derivatives where a phosphate proton is substituted with an amino-alkyl group, sugar derivatives where a phosphate proton is substituted with a sugar such as glucose.

The term “amino-alkyl group” refers to a group comprising an amino (—NH₂) group and an alkyl group. The term “alkyl” refers to straight chain, branched chain or cyclic hydrocarbon groups having from 1 to 8 carbon atoms. Examples include methyl, ethyl, propyl, isopropyl, butyl, isobutyl, sec-butyl, tert-butyl, pentyl, hexyl, cyclohexyl, heptyl, and octyl. Phosphatidyl choline derivatives are most preferred.

The phosphate compounds of tocopherol may be selected from the group consisting of mono-(tocopheryl)phosphate, mono-(tocopheryl)phosphate monosodium salt, mono-(tocopheryl)phosphate disodium salt, mono-(tocopheryl)phosphate monopotassium salt, mono-(tocopheryl)phosphate dipotassium salt, di-(tocopheryl)phosphate, di-(tocopheryl)phosphate monosodium salt, di-(tocopheryl)phosphate monopotassium salt, or a mixture thereof. These phosphate compounds may be derived from the alpha, beta, gamma or delta form of tocopherol, or a combination thereof.

When a mixture of a mono-phosphate ester and a di-phosphate ester, that is a mono-(tocopheryl)phosphate and di-(tocopheryl)phosphate (which may in some instances herein be simply referred to as “TPM”), the ratio is preferably at least 2:1, more preferably within the range of about 4:1 to about 1:4, most preferably within the range of about 6:4 to about 8:2. The ratio may be about 6:4 or about 8:2.

The matrix layer may comprise a phosphate compound of tocopherol in an amount within the range of about 0.01% w/w to about 10% w/w, about 0.1% w/w to about 5% w/w, about 0.5% w/w to about 2% w/w or to about 3% w/w, of the total concentration of the matrix layer. In one embodiment, the phosphate compound of tocopherol is present in an amount of about 1% w/w to about 1.5% w/w of the total concentration of the matrix layer.

### Polymer Carrier

The composition, or matrix layer, also comprises a polymer carrier.

The polymer carrier may comprise natural and synthetic polymers, co-polymers, or terpolymers.

Natural polymers include rubbers, elastomers, polysaccharides such as cellulose, natural resins such as shellac and amber.

Synthetic polymers include, for example, polyacrylates, polyamides, polyesters, polycarbonates, polyimides, polystyrenes, acrylonitrile butadiene styrene, polyacrylonitrile, polybutadiene, poly(butylene terephthalate), poly(ether sulphone), poly(ether)ketones, polyethylene, poly(ethylene glycol), poly(ethylene terphthalate), polypropylene, polytetratfluroethylene, styrene-acrylonitrile resin, poly(trimethylene terephthalate), polyurethanes, polyvinyl butyral, polyvinylchlorides, polyvinylidenedifluoride, poly(vinyl pyrrolidone), polychloroprene, fluoroelastomers, chloro-sulphonated rubbers, hypromellose, polyolefine elastomer, polyacrylamide, chlorinated polyethylene, polyethersulphone, nylon, liquid crystal polymers, polyethylene terephthalate (PET), polyphenylsulphone, polypthalaminepolyvinyl alcohol derivatives, poly ethylene glycols, ethylene vinyl acetate, polymethyl methacrylate, cellulose derivatives such as ethyl cellulose, hydroxyl propyl methyl cellulose, sugar derivatives (gums) including derivatives of sorbitol and mannitol, and silicone oil derivatives such as polysiloxanes.

Preferred polymer carriers that are suitable for use in the matrix layer of the present invention include polyvinyl pyrrolidone (e.g. PVP K90, MW 360,000 Da), polysiloxanes and polymethyl methacrylate (e.g. Eudragit E100).

The polymer carrier used in the matrix layer may have sufficient tackiness to enable the matrix patch to adhere to skin. For instance, amine-resistant polysiloxanes and mixtures thereof can be used in the matrix layer. A mixture of a polysiloxane of medium tack and a polysiloxane of high tack is used would be most suitable. The polysiloxanes may be synthesized from linear bifunctional and branched polyfunctional oligomers. It has been found that the ratio of both types of oligomers determines the physical properties of the polymers. More polyfunctional oligomers result in a more cross-linked polymer with a higher cohesion and a reduced tack, less polyfunctional oligomers result in a higher tack and a reduced cohesion. A high tack version should be tacky enough for the matrix patch to adhere to the surface of skin. A medium tack version, on the other hand, may not be tacky at all but could be useful by providing a softening effect to other components included in the matrix layer. To increase the adhesive power of the matrix layer, a silicone oil (e.g. dimethicone) could be added.

The matrix layer may comprise a polymer carrier in an amount of from about 20% w/w up to about 90% w/w, from about 30% w/w up to about 80% w/w, from about 55% w/w up to about 65% w/w, of the total weight of the matrix layer.

The polymer carrier may also comprise inert carrier components, such as for example, anti-tacking agents, tackifiers, and plasticizers to achieve appropriate softness, flexibility and “tackiness” for the polymer carrier to enable the matrix layer to adhere to the surface of skin, and thus provide consistent delivery.

For polymers which are naturally “tacky” and may need anti-tackiness to have an appropriate consistency, anti-tacking agents that are solid with no stickiness property (i.e. low ability to retain solvents upon drying) and that can be mixed well (i.e. do not crystallise upon drying) with the polymer carrier may be suitable. The selection would be based on the polymer-type. Many surfactants are suitable for use as an anti-tacking agent with a polymer carrier. A more specific example of an anti-tacking agent is succinic acid.

In order to enhance the ability of the matrix layer to adhere to the surface of skin, it may optionally contain a tackifier (or tacking agent). Tack can be controlled by combining adhesives of varying hardnesses (glass temperature or T_{g}). Typically, a tackifier is a polymer which is insoluble in water and composed of a monomer which contains partly or wholly a (meth)acrylic alkyl ester. Such types of polymers include, but are not limited to, acrylic, N-butyl-methacrylic copolymer (Primal N580NF, sold by Japan Acrylic Chemical Company, Ltd.), acrylic methyl, acrylic 2-ethylhexyl copolymer (Nikasol TS-6520, sold by Nippon Carbide Industries Company, Ltd), polyacrylic acid (Jurymer AC-IOLPH, sold by Nihon Junyaku Company, Ltd), methacrylic copolymer L (Plastoid L50, sold by Rohm Pharma GmbH), and aminoalkylmethacrylate copolymer E (Plastoid E35L, Plastoid E35M, Plastoid E35H, all sold by Rohm Pharma GmbH). Other non-limiting examples include rosin esters, hydrogenated rosins, dipropylene glycol dibenzoate, and/or mixed hydrocarbons, and acrylic copolymers (e.g. Flexbond 150 adhesive by Air Products).

Plasticizers are additives that increase the plasticity or fluidity of the material to which they are added. Plasticizers may be used in the present invention to soften the final product increasing its flexibility and making it less brittle. Suitable plasticizers include phthalates, esters of polycarboxylic acids with linear or branched aliphatic alcohols of moderate chain length, acetylated monoglycerides, alkyl citrates, triethyl citrate (TEC), acetyl triethyl citrate (ATEC), tributyl citrate (TBC), acetyl tributyl citrate (ATBC), trioctyl citrate (TOC), acetyl trioctyl citrate (ATOC), trihexyl citrate (THC), acetyl trihexyl citrate (ATHC), butyryl trihexyl citrate (BTHC, trihexyl o-butyryl citrate), trimethyl citrate (TMC), alkyl sulphonic acid phenyl ester, bis(2-ethylhexyl)phthalate (DEHP), bis(n-butyl)phthalate (DnBP, DBP), diisooctyl phthalate (DIOP), bis(n-butyl)phthalate (DnBP, DBP), diisobutyl phthalate (DIBP), bis(2-ethylhexyl)adipate (DEHA), dimethyl adipate (DMAD), monomethyl adipate (MMAD), dioctyl adipate (DOA), dibutyl sebacate (DBS), dibutyl maleate (DBM), diisobutyl maleate (DIBM), benzoates, epoxidized vegetable oils, N-ethyl toluene sulfonamide (o/p ETSA), N-(2-hydroxypropyl)benzene sulfonamide (HP BSA), N-(n-butyl)benzene sulfonamide (BBSA-NBBS), tricresyl phosphate (TCP), tributyl phosphate (TBP), triethylene glycol dihexanoate (3G6, 3GH), tetraethylene glycol diheptanoate (4G7), and polyvinylpyrrolidone. Dibutyl sebacate (DBS) is a preferred plasticizer.

Inert carrier components may be present in the matrix layer in an amount of from 0.001% w/w up to about 50% w/w, up to about 40% w/w, up to about 30% w/w, of the total weight of the matrix layer. In one embodiment, the matrix layer comprises an anti-tacking agent (such as succinic acid) and a plasticizer (such as dibutyl sebacate) in a total amount of about 35% w/w of the total weight of the matrix layer.

The amount of polymer carrier and optional inert carrier components present in the matrix layer will depend on the specific opioid to be administered. Generally, however, the matrix layer may comprise these components in an amount of from about 50% w/w up to about 99% w/w, from about 80% w/w up to about 98% w/w, from about 90% w/w up to about 98% w/w, of the total weight of the matrix layer. In one embodiment, the matrix layer comprises these components in the amount of about 95% w/w of the total weight of the matrix layer.

It should be noted that, in some instances herein, the term “polymer carrier” could be used collectively to refer to the polymer carrier and the inert carrier components.

### Additional Optional Components

The matrix layer may optionally further comprise one or more excipients (in addition to the inert carrier components discussed above).

A person skilled in the art of the invention would appreciate what are suitable excipients for inclusion in the matrix layer of the invention. Some examples include, but are not limited to, solvents, thickeners or gelling agents, preservatives, surfactants, stabilizers, plasticizers, adhesives or glues, buffers, emollients, colours, fragrances, and appearance modifiers. It will be appreciated that any excipients which have been approved for use in pharmaceutical products by the regulatory bodies may be employed in the matrix layers (or compositions) of the present invention. The amount of a particular excipient or excipients to be used in a matrix layer of the present invention would also be appreciated by a person skilled in the art.

### Opioids

It has been surprisingly found that the transdermal delivery patch of the present invention can effectively administer opioids.

An opioid is a chemical that works by binding to opioids receptors, which are found principally in the central nervous system and the gastrointestinal tract. The receptors in these two organ systems mediate both the beneficial effects and the side effects of opioids.

There are a number of broad classes of opioids: natural opiates which are alkaloids contained in the resin of the opium poppy such as morphine, codeine and thebaine; semi-synthetic opioids created from natural opiates such as hydromorphone, hydrocodone, oxycodone, oxymorphone, desomorphine, diacetylmorphine (heroin), nicomorphine, dipropanoylmorphine, benzylmorphine and ethylmorphine; fully synthetic opioids such as fentanyl, pethidine, methadone, tramadol and dextropropoxyphene; and, endogenous opioid peptides, produced naturally in the body, such as endorphins, enkephalins, dynorphins, and endomorphins.

Opioids produce an analgesic effect, generally through their interaction with opioid receptors. Opioid analgesics may be opioid receptor agonists, opioid receptor partial agonists, opioid antagonist or opioid receptor mixed agonist-antagonists.

Opioid receptor agonists include, but are not limited to, morphine, depomorphine, etorphine, heroin, hydromorphone, oxymorphone, levorphanol, methadone, levomethadyl, meperidine, fentanyl, sufentanyl, alfentanil, codeine, hydrocodone, oxycodone, and mixtures of the foregoing.

Opioid receptor antagonists include, but are not limited to, naloxone and naltrexone.

An opioid receptor mixed agonist-antagonist has mixed opioid agonist/antagonist activities, or one that exhibits only partial agonist activity. Compounds which exhibit mixed agonist/antagonist activity include, but are not limited to, buprenorphine, nalbuphine, butorphanol, pentazocine, and mixtures of such compounds. Compounds which exhibit partial agonist activity include, but are not limited to ethylketocyclazocine.

The present invention is not limited to the delivery of a single opioid: embodiments of the invention may include mixtures of opioids. For the avoidance of any doubt, it is to be noted that the singular forms “a”, “an” and “the” should be read as encompassing plural forms, unless the context clearly indicates otherwise.

The present invention is also not limited to the specific opioid compounds mentioned herein: pharmaceutically-acceptable salts, prodrugs, and other derivatised compounds are envisioned as well.

The present invention is further not limited solely to the administration of opioids: other therapeutic compounds may be incorporated into the matrix layer in addition to the opioid, such as for example, steroidal and non-steroidal anti-inflammatory agents, local anaesthetics and/or antibiotics.

Examples of “opioids” include, but are not limited to: Opium alkaloids including Phenanthrenes naturally occurring in opium such as codeine, morphine, thebaine and oripavine (the active metabolite of thebaine); Synthetic derivatives such as diacetylmorphine (heroin), dihydrocodeine, hydrocodone, hydromorphone, nicomorphine, desmorphine, ethylmorphine, dipropanoylmorphine, oxycodone and oxymorphone; Synthetic opioids including Anilidopiperidines such as fentanyl, alphamethylfentanyl, alfentanil, sufentanil, remifentanil, carfentanyl and ohmefentanyl, Phenylpiperidines such as pethidine (meperidine), ketobemidone, MPPP, allylprodine, prodine and PEPAP, Diphenylpropylamine derivatives such as propoxyphene, dextropropoxyphene, dextromoramide, bezitramide, piritramide, methadone, dipipanone, levomethadyl acetate (LAAM), difenoxin, diphenoxylate and loperamide, Benzomorphan derivatives such as dezocine, pentazocine and phenazocine, Oripavine derivatives such as buprenorphine, dihydroetorphine and etorphine, Morphinan derivatives such as butorphanol, nalbuphine, levorphanol and levomethorphan, and others such as lefetamine, meptazinol, tilidine, tramadol and tapentadol; Opioid receptor antagonists including nalmefene, naloxone and naltrexone; and pharmaceutically-acceptable salts, prodrugs, or derivatised compounds thereof.

In a preferred embodiment, the opioid is oxycodone or dihydrohydroxycodeinone (oxycodone base).

The opioid may be present in a therapeutically effective amount, that is, an amount necessary to achieve a desired therapeutic effect. Typically, the opioid will be present in an amount of from about 0.1% w/w up to about 30% w/w, up to about 20% w/w, up to about 10% w/w, of the total concentration of the matrix layer. In one embodiment, the matrix layer will have an opioid concentration of about 4.5% w/w to about 5.5% w/w of the total concentration of the matrix layer.

### Preparation of the Matrix Patch

The matrix patch of the present invention may be prepared by a variety of techniques.

One technique involves combining the polymer carrier and any inert carrier components such as an anti-tacking agent and/or plasticizer with a suitable solvent. This is combined with a dispersion comprising the opioid and the phosphate compound of tocopherol, and is stirred until complete homogenisation is achieved. The composition may then be placed in a suitable mould and dried. In a preferred method, the composition may be dried by heating up to about 90° C., preferably for 0.5 to 24 hours. However, formulating and/or drying may be conducted at a temperature within the range of about 30° C. to about 90° C. It has been found that formulating and/or drying at a temperature of about 75° C. results in better delivery of the opioid.

The ratio of components in of this composition, polymer carrier to opioid to phosphate compound of tocopherol, is preferably at least 10:5:1, 14:5:1 or 14:10:2. The opioid:TP ratios may be between about 5:5 to about 5:0.5, with the most preferred value of about 5:1. The polymer carrier:[opioid and TP] is about 1:1 to about 3:1, with preferred values of about 7:6 to about 7:3.

The composition comprising the phosphate compound of tocopherol and the polymer carrier is suitable for use in a transdermal delivery patch for administration of an opioid. This composition essentially forms the matrix layer in a transdermal delivery patch. The matrix layer may be a solid or semi-solid layer.

The transdermal delivery patch usually would also comprise a backing layer. The backing layer acts as a support or substrate for the matrix layer. When preparing a matrix patch using a mould, the backing layer would be placed in the mould before addition of the matrix layer composition.

Accordingly, the matrix layer essentially has two surfaces: a first surface and a second surface opposite the first surface, where the first surface is in contact with the backing layer and the second surface being adapted to be in diffusional contact with the skin of a subject. The subject may be a human or animal.

The present invention therefore also provides use of a matrix patch for transdermal delivery of an opioid, the matrix patch comprising (i) a backing layer, and (ii) a matrix layer which comprises a phosphate compound of tocopherol, a polymer carrier, and an opioid.

Preferably, the backing layer is occlusive or impermeable to protect the matrix layer from the outer environment. However, a non-occlusive backing layer could also be used, so long as the packaging of the matrix patch is fully occlusive to prevent degradation of the matrix layer. An occlusive backing layer is preferred.

The backing layer may be of any thickness, however in the art, backing layers typically have a thickness of about 0.0005 inches to about 0.01 inches.

The present invention therefore provides a transdermal delivery patch for administration of an opioid comprising (i) a backing layer, and (ii) the matrix layer which comprises a phosphate compound of tocopherol, a polymer carrier, and an opioid.

The matrix patch may further comprise a liner which is a removable protective or impermeable layer, usually but not necessarily rendered “non-stick” so as not to stick to the matrix layer. The liner, which may also be referred to as the release liner, protects the matrix patch during storage. During use, the release liner is to be removed.

The liner may be made from the same material as the backing layer, however it may also be a metal foil, Mylar (registered trademark), polyethylene terephthalate, siliconized polyester, fumed silica in silicone rubber, polytretrafluoroethylene, cellophane, siliconized paper, aluminized paper, polyvinyl chloride film, composite foils or films containing polyester such as polyester terephthalate, polyester or aluminized polyester, polytetrafluoroethylene, polyether block amide copolymers, polyethylene methyl methacrylate block copolymers, polyurethanes, polyvinylidene chloride, nylon, silicone elastomers, rubber-based polyisobutylene, styrene, styrene-butadiene, and styrene-isoprene copolymers, polyethylene, and polypropylene.

The release liner may be of any thickness, however in the art, release liners typically have a thickness of about 0.01 mm to about 2 mm.

The matrix patch may also comprise an adhesive layer. The adhesive layer may be an additional layer to the matrix layer, or may be included on the outer margin of the backing layer where the backing layer extends beyond the edges of the matrix layer. Polymeric adhesives useful for transdermal patches include polyacrylate polymers, rubber-based adhesives and polysiloxane adhesives. These types of materials, as well as others, are described by Van Norstrand (The Handbook of Pressure Sensitive Adhesive Technology Second Edition 1989), which is hereby incorporated by reference. Examples of commercially available adhesives include, but are not limited to, polyacrylate adhesives sold under the trademarks DUROTAK (registered trademark) by National Starch and Chemical Corporation, Bridgewater, N.J., as well as GELVA-MULTIPOLYMER SOLUTION (registered trademark) by Cytek Surface Specialties, Smyrna, Ga.

### Advantages

It has surprisingly been found that opioids can be effectively administered using a transdermal delivery patch comprising a matrix layer which comprises a phosphate compound of tocopherol and a polymer carrier.

Transdermal delivery options for include, for example, topical creams and gels, and skin patches.

Creams and gels may present difficulties with compliance and dosage control, and may be considered messy or unpleasant by patients.

There are different forms of skin patches, including “reservoir” patches and “matrix” patches. Patches may also be single- or multi-layered. A “reservoir” patch essentially has a liquid or gel compartment containing the drug solution or suspension separated by a membrane and a layer of adhesive. In a “matrix” patch, the drug dispersion is present in a semi-solid or solid layer, which may or may not also comprise the adhesive material.

Reservoir patches overcome some of the dosage difficulties with topical creams and gels, however the delivery may be uneven or inconsistent, and there is some risk of perforation of the reservoir. An additional issue relates to delivery of prescribed drugs which may be addictive and subject to abuse. Gels, creams and reservoir patches provide limited barriers to extraction of the drug substance, whereas incorporation of the drug substance within a matrix layer represents a significant, if not almost impossible barrier to extraction of the drug substance.

Delivery of an active orally or by injection typically results in a delivery profile which is non-linear. Transdermal delivery provides a non-invasive way of potentially achieving sustained steady state delivery.

Without wishing to be bound by theory, the presence of a phosphate compound of tocopherol may reduce any skin irritation caused by the opioid and enhance the skin permeation of the opioid. It has also been found that the components of the matrix layer do not formulate well together without the presence of a phosphate compound of tocopherol.

### FIGURES

The examples will be described with reference to the accompanying figures in which:

FIG. 1 is a schematic diagram of a matrix patch of one embodiment of the present invention;

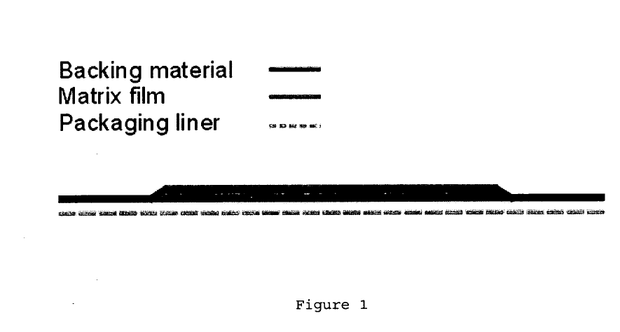

FIG. 2 is a graph comparing the delivery of oxycodone using a matrix patches of the present invention prepared with different drying regimes;

FIG. 3 is a graph comparing the delivery of oxycodone using matrix patches of the present invention prepared with and without a glue layer;

FIG. 4 is a graph comparing the delivery of oxycodone using matrix patches of the present invention prepared with and without an occlusive backing layer;

FIG. 5 is a graph showing the results of pharmacokinetic testing conducted after application of matrix patches of the present invention; and

FIG. 6 is a graph showing the results of pharmacodynamic testing conducted after application of matrix patches of the present invention.

### EXAMPLES

Various embodiments/aspects of the present invention will now be described with reference to the following non-limiting examples.

### Example 1

### Manufacture of Matrix Patch

Final matrix layer composition

| | Percentage by |
|---|---|
| Components | weight, after drying |
| A mixture of mono-(tocopheryl) phosphate | 1.1% w/w |
| and di-(tocopheryl) phosphate in a ratio of 6:4 | |
| Oxycodone | 5.5% w/w |
| Eudragit E100 (polymethyl methacrylate) | 60.6% w/w |
| Dibutyl sebacate | 27.3% w/w |
| Succinic acid | 5.5% w/w |

### Small Scale Laboratory Manufacturing

The components were dissolved in a solvent solution (acetone:isopropanol:ethyl alcohol 60:6.6:33.5 by weight). The resulting solution was then poured into individual casts (containing suitable backing layers) at room temperature and the solvent was allowed to evaporate at 75° C. for 1.5 hours.

### Large Scale Manufacturing

All matrix layer components could be combined at a suitable temperature to produce a homogeneous molten mass. The molten mass can then be cast on a cold surface (for example, a rotating mill with a suitable backing layer, or sheet, thereon) and allowed to solidify. Individual matrix patches of varying sizes may then be cut.

In both methods, the matrix layer would be relatively thin; however, the thickness of the matrix layer can be varied depending on the desired properties of the matrix patch.

### Example 2

### Alterbate Method for Manufacture of Matrix Patch

Matrix patches were constructed by dissolving 20% w/w solid mixture of Eudragit E100 granules, dibutyl sebacate, succinic acid (the components other than TPM and oxycodone in the matrix layer may collectively be referred to as the “polymer carrier”); a mixture of mono-(tocopheryl)phosphate and di-(tocopheryl)phosphate in a ratio of 6:4 (TPM); and oxycodone base in 60:6.6:33.4w/w acetone/isopropyl alcohol/ethyl alcohol. The mixture was then transferred into 6cm² circular aluminium cast-lined on the underside with polyester backing (1.66 mil, 3M Scotchpak™, 3M, MN) and the solvent evaporated in an oven at either 45° C. overnight or 75° C. for 1.5 hours. Where glue was used, the glue was Duro-Tak adhesive and in this example succinic acid was omitted from the formulation.

TABLE 1
Composition, excipient ratios and manufacture
conditions of matrix patches

| | | Oxy- | Vol. | | | |
|---|---|---|---|---|---|---|
| Patch | Ratio | codone | stock | Dry temp./ | Succinic | |
| No. | (PC:O:TPM)* | (mg) | (ml) | time | acid | Glue |
| 1 | 10:5:1 | 10 | 2 | 45° C. | Yes | No |
| | | | | overnight | | |
| 2 | 14:5:1 | 10 | 2 | 45° C./ | Yes | No |
| | | | | overnight | | |
| 3 | 14:5:1 | 5 | 1 | 45° C./ | Yes | No |
| | | | | overnight | | |
| 4 | 14:5:1 | 5 | 1 | 75° C./1.5 h | Yes | No |
| 5 | 14:10:2 | 5 | 0.5 | 75° C./1.5 h | No | Yes |

*Refers to ratio of polymer carrier:oxycodone:TPM

### Example 3

### Comparative Testing for Drying Temperatures

Oxycodone matrix patches were made according to Example 1 (small scale) above, testing the variable of the two different heating regimes. The matrix patches were adhered to full thickness human skin applied to a Franz cell with PBS as the receiver solution. Time points were taken at 18, 22, 24, 42, 44, 68 and 75 hours and the receiver solution was tested by HPLC to determine the concentration of oxycodone which had passed through the skin.

TABLE 2
Parameters in the patches tested

| | | | Vol. | Dry | | |
|---|---|---|---|---|---|---|
| | Ratio | Oxycodone | stock | temp./ | Succinic | |
| Patch | (PC:O:TPM)* | (mg) | (ml) | time | acid | Glue |
| A | 14:5:1 | 10 | 2 | 45° C./ | Yes | No |
| | | | | over- | | |
| | | | | night | | |
| B | 14:5:1 | 10 | 2 | 75° C./ | Yes | No |
| | | | | 1.5 h | | |

*Refers to ratio of polymer carrier:oxycodone:TPM

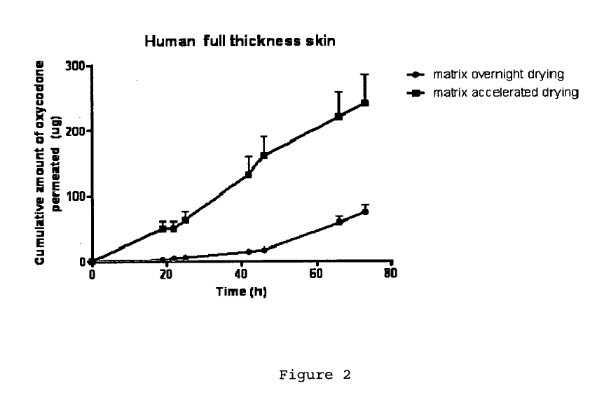

The results outline in FIG. 2 show that the matrix patch manufactured using the higher (accelerated) drying temperature has increased transdermal delivery properties compared with the matrix patch manufactured with drying at a lower temperature.

### Example 4

### Comparative Testing to Determine Effect of an External Glue Layer

Matrix patches were manufactured and the receiver solution tested as in Example 3, with testing time points of 0.5, 1, 3, 4 and 20 hours.

TABLE 3
Parameters in the patches tested

| | | | Vol. | Dry | | |
|---|---|---|---|---|---|---|
| Patch | Ratio | Oxycodone | stock | temp./ | Succinic | |
| No. | (PC:O:TPM)* | (mg) | (ml) | time | acid | Glue |
| C | 14:5:1 | 10 | 2 | 75° C./ | Yes | No |
| | | | | 1.5 h | | |
| D | 14:5:1 | 10 | 2 | 75° C./ | No | Yes |
| | | | | 1.5 h | | |

*Refers to ratio of polymer carrier:oxycodone:TPM

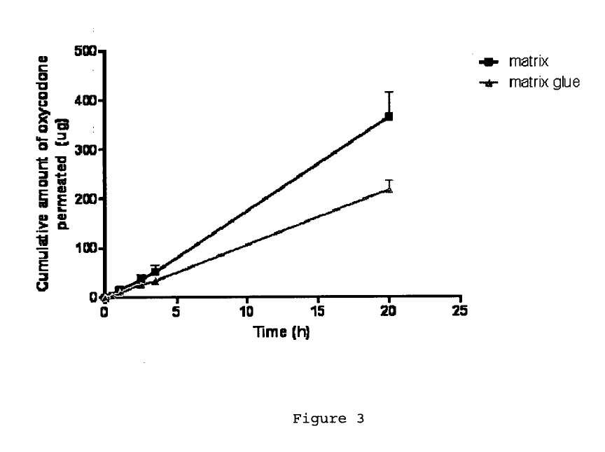

The results of this comparison outlined in FIG. 3 clearly demonstrate that using a matrix patch which includes an adhesive layer results in reduced transdermal penetration of the oxycodone compared with the matrix patches formulated to be self-adhesive.

### Example 5

Comparative Testing to Determine Effect of an Occlusive Backing Layer Compared with No Backing Layer

The matrix patches were manufactured and the receiver solution tested as in Examples 3 and 4, at time points 1, 2, 3, 4 and 5 hours.

TABLE 4
Parameters in the patches tested

| | | Oxy- | Vol. | | | |
|---|---|---|---|---|---|---|
| Patch | Ratio | codone | stock | Dry temp./ | Occlusive | |
| No. | (PC:O:TPM)* | (mg) | (ml) | time | backing | Glue |
| E | 14:5:1 | 10 | 2 | 75° C./1.5 h | Yes | No |
| F | 14:5:1 | 10 | 2 | 75° C./1.5 h | No | No |

*Refers to ratio of polymer carrier:oxycodone:TPM

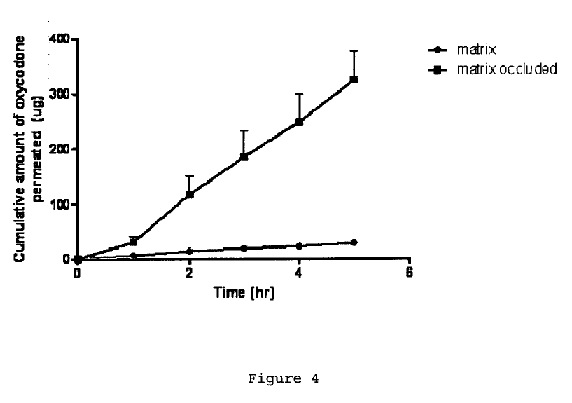

The results outlined in FIG. 4 clearly show that the penetration of the oxycodone transdermally is far superior when an occlusive backing layer is used with the patch compared with a patch without the adhesive backing layer.

### Example 6

### Pharmacokinetic Testing

This example compares plasma PK parameters using Patch Nos. 1, 2, 4 and 5 from Example 2.

Matrix patches were cut from the polyester backing and adhered to the shaved and washed back of a 10-12 week old male Sprague-Dawley rat with a 6×7 cm Tegaderm HP™ (3M, MN) adhesive dressing either with the backing layer in place or removed (see Table 5). Tegaderm serves to hold the occlusive backing layer in place, or if the backing layer is absent, holds the matrix patch itself in place.

The day after the matrix patches were adhered to the shaved section, blood samples removed from the tail tip following ˜1 mm tip amputation at specified times. The PK parameters quantified were:

Cₘₐₓ: the maximal observed plasma oxycodone concentration.

AUC₀₋₄: The area under the curve between 0 and 4 hours (the duration of the experiment was 4 hours) and is a measure of the total amount of drug delivered.

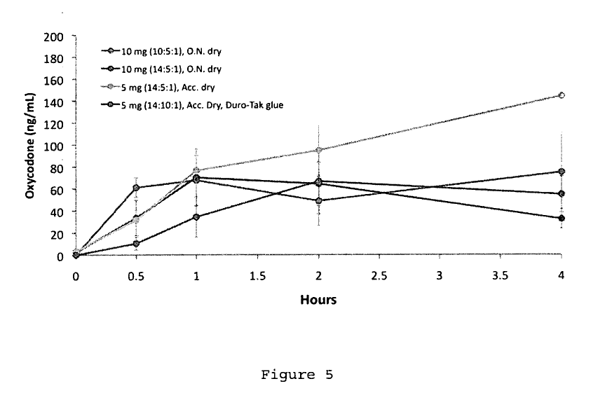

The results in FIG. 5 and Table 5 demonstrate that the matrix patches of the present invention in various formulations are able to effectively deliver the oxycodone to the rats as demonstrated by the pharmacokinetic data.

TABLE 5
Estimated pharmacokinetic parameters
of rats administered matrix patches

| | Oxycodone | | | | |
|---|---|---|---|---|---|
| Patch | dose | | | Cₘₐₓ | AUC₀₋₄ |
| No. | (mg/kg) | Occlusive | n | (ng/mL) | (ng · mL/min) |
| 1 | 41.8 ± 0.4 | No | 17 | 93 ± 16 | 13681 ± 2367 |
| 2 | 45.0 ± 2.1 | Yes | 9 | 92 ± 27 | 11959 ± 2910 |
| 4 | 21.7 ± 0.1 | Yes | 5 | 144 ± 33 | 21637 ± 5189 |
| 5 | 18.1 ± 0.3 | Yes | 5 | 74 ± 29 | 11161 ± 4636 |

‘n’ = no. of animals

### Example 7

### Pharmacodynamic Testing

Rats were prepared and dosed similar to Example 6 using Patch Nos. 1, 3 and 5 from Example 2.

The day after the matrix patches were adhered to the shaved section, antinociception of the hind-paw was assessed with a plantar analgesiometer with the IR source calibrated to 190 Mu/cm².

The following PD parameters were assessed:

Maximum: The maximum time it took for the rat to remove its paw in response to the heat stimulus. The higher the number, the longer it took for the rat to respond and the deeper the oxycodone induced analgesia.

AUC: This is a measure of the total analgesia over the observation period as measured by the area under the curve between 0 and 4 hour, and is useful for comparing the response to different treatments.

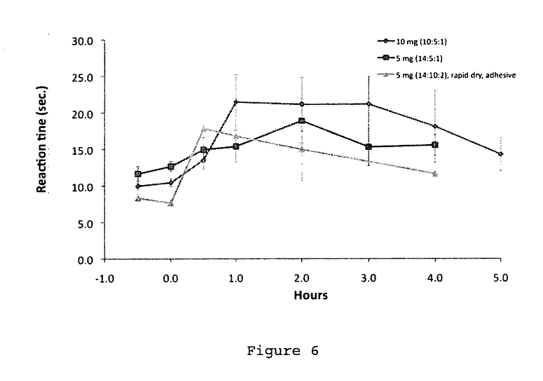

The baseline response time is indicated in FIG. 6 at t=(−0.5 h) and t=0.

The results outlined in Table 6 below and FIG. 6 demonstrate that analgesia was effectively administered to the rats using a variety of compositions of the present invention.

TABLE 3
Pharmacodynamic parameters from rats
administered different matrix patches

| | Oxycodone | | | |
|---|---|---|---|---|
| Patch | dose | | Max | AUC₀₋₄ |
| No. | (mg/kg) | n | (sec.) | (sec/h) |
| 1 | 41.0 ± 0.8 | 5 | 20.7 ± 3.5 | 57.6 ± 9.1 |
| 3 | 21.8 ± 0.6 | 5 | 22.3 ± 3.3 | 76.8 ± 13.1 |
| 5 | 21.6 ± 0.5 | 4 | 20.5 ± 2.3 | 64.0 ± 6.4 |

‘n’ = no. of animals

In this specification, except where the context requires otherwise, the words “comprise”, “comprises”, and “comprising” mean “include”, “includes”, and “including” respectively, i.e. when the invention is described or defined as comprising specified features, various embodiments of the same invention may also include additional features.

Although this invention has been described by example and with reference to possible embodiment thereof, it is to be understood that modifications or improvements may be made thereto without departing from the scope of the invention.

1. A composition suitable for use in a transdermal delivery patch for administration of an opioid, the composition comprising a phosphate compound of tocopherol and a polymer carrier.

2. The composition of claim 1, wherein the transdermal delivery patch is a matrix patch.

3. The composition of claim 1, wherein the phosphate compound of tocopherol is selected from the group consisting of mono-(tocopheryl)phosphate, mono-(tocopheryl)phosphate monosodium salt, mono-(tocopheryl)phosphate disodium salt, mono-(tocopheryl)phosphate monopotassium salt, mono-(tocopheryl)phosphate dipotassium salt, di-(tocopheryl)phosphate, di-(tocopheryl)phosphate monosodium salt, di-(tocopheryl)phosphate monopotassium salt, or a mixture thereof.

4. The composition of claim 3, wherein the phosphate compound of tocopherol is present in an amount within the range of about 0.01% w/w to about 10% w/w, about 0.1% w/w to about 5% w/w, or about 0.5% w/w to about 2% w/w or to about 3% w/w, of the total concentration of the composition.

5. The composition of claim 4, wherein the phosphate compound of tocopherol is present in an amount of about 1% w/w to about 1.5% w/w of the total concentration of the composition.

6. The composition of claim 1, wherein the polymer carrier comprises natural and synthetic polymers, co-polymers, or terpolymers.

7. The composition of claim 6, wherein the polymer carrier comprises polyvinyl pyrrolidone, polysiloxanes or polymethyl methacrylate.

8. The composition of claim 6, wherein the polymer carrier is present in an amount of from about 20% w/w up to about 90% w/w, from about 30% w/w up to about 80% w/w, or from about 55% w/w up to about 65% w/w, of the total weight of the composition.

9. The composition of claim 6, wherein the polymer carrier also comprises an inert carrier component selected from the group consisting of anti-tacking agents, tackifiers, and plasticizers.

10. The composition of claim 9, wherein the inert carrier components is present in an amount of from 0.001% w/w up to about 50% w/w or up to about 40% w/w or up to about 30% w/w, of the total weight of the composition.

11. Use of a phosphate compound of tocopherol and a polymer carrier as a matrix layer in a transdermal delivery patch for administration of an opioid.

12. A transdermal delivery patch for administration of an opioid comprising (i) a backing layer, and (ii) a matrix layer, which comprises a composition of claim 1, and an opioid.

13. The transdermal delivery patch of claim 12, wherein the opioid is selected from the group consisting of morphine, codeine or thebaine; hydromorphone, hydrocodone, oxycodone, oxymorphone, desomorphine, diacetylmorphine (heroin), nicomorphine, dipropanoylmorphine, benzylmorphine or ethylmorphine; fentanyl, pethidine, methadone, tramadol or dextropropoxyphene; endorphins, enkephalins, dynorphins, or endomorphins.

14. The transdermal delivery patch of claim 12, wherein the opioid is selected from the group consisting of opioid receptor agonists including morphine, depomorphine, etorphine, heroin, hydromorphone, oxymorphone, levorphanol, methadone, levomethadyl, meperidine, fentanyl, sufentanyl, alfentanil, codeine, hydrocodone, oxycodone, and mixtures thereof; opioid receptor antagonists including naloxone and naltrexone; opioid receptor mixed agonist-antagonists including buprenorphine, nalbuphine, butorphanol, pentazocine, and mixtures thereof; and, ethylketocyclazocine.

15. The transdermal delivery patch of claim 12, wherein the opioid is selected from the group consisting of codeine, morphine, thebaine and oripavine; diacetylmorphine (heroin), dihydrocodeine, hydrocodone, hydromorphone, nicomorphine, desmorphine, ethylmorphine, dipropanoylmorphine, oxycodone and oxymorphone; fentanyl, alphamethylfentanyl, alfentanil, sufentanil, remifentanil, carfentanyl and ohmefentanyl; pethidine (meperidine), ketobemidone, MPPP, allylprodine, prodine and PEPAP; propoxyphene, dextropropoxyphene, dextromoramide, bezitramide, piritramide, methadone, dipipanone, levomethadyl acetate (LAAM), difenoxin, diphenoxylate and loperamide; dezocine, pentazocine and phenazocine; buprenorphine, dihydroetorphine and etorphine; butorphanol, nalbuphine, levorphanol and levomethorphan; lefetamine, meptazinol, tilidine, tramadol and tapentadol; nalmefene, naloxone and naltrexone; and pharmaceutically-acceptable salts, prodrugs, or derivatised compounds thereof.

16. The transdermal delivery patch of claim 12, wherein the opioid is oxycodone or dihydrohydroxycodeinone (oxycodone base).

17. The transdermal delivery patch of claim 12, wherein the opioid is present in an amount of from about 0.1% w/w up to about 30% w/w or up to about 20% w/w or up to about 10% w/w, of the total concentration of the matrix layer.

18. The transdermal delivery patch of claim 12, wherein the opioid is present in an amount of about 4.5% w/w to about 5.5% w/w of the total concentration of the matrix layer.

19. The trandermal delivery patch of claim 12, wherein the backing layer is occlusive.

20. Use of a matrix patch for transdermal delivery of an opioid, the matrix patch comprising (i) a backing layer and (ii) a matrix layer, which comprises a composition of claim 1, and an opioid.

21. A method for preparing a transdermal delivery patch for administration of an opioid comprising the steps of:
(i) combining a polymer carrier and optional inert carrier components with a suitable solvent;
(ii) combining (i) with a dispersion comprising a phosphate compound of tocopherol and an opioid;
(iii) stirring (ii) until complete homogenisation is achieved;
(iv) placing (iii) in a mould comprising a suitable backing layer; and
(v) drying the compositions in the mould by heating them up to about 90° C. for about 0.5 to about 24 hours.

22. The method of claim 21, wherein drying is conducted at a temperature of 75° C.
